(19) United States
(12) Patent Application Publication
(10) Pub. No.: US 2025/0087336 A1
(43) Pub. Date: Mar. 13, 2025

(54) MULTI-MODAL MULTI-VIEW CLASSIFICATION METHOD FOR ECHOCARDIOGRAMS BASED ON DEEP LEARNING ALGORITHM

(71) Applicant: UNION HOSPITAL TONGJI MEDICAL COLLEGE HUST, Wuhan City (CN)

(72) Inventors: Li ZHANG, Wuhan City (CN); Ye ZHU, Wuhan City (CN); Mingxing XIE, Wuhan City (CN); Zisang ZHANG, Wuhan City (CN); Yiwei ZHANG, Wuhan City (CN); Chun WU, Wuhan City (CN); Manwei LIU, Wuhan City (CN); Ziming ZHANG, Wuhan City (CN); Shuangshuang ZHU, Wuhan City (CN); Sushan XIAO, Wuhan City (CN); Chunyan XU, Wuhan City (CN); Junmin ZHANG, Wuhan City (CN); Wenwen CHEN, Wuhan City (CN); Zubaire YASHENG, Wuhan City (CN)

(21) Appl. No.: 18/585,805

(22) Filed: Feb. 23, 2024

(30) Foreign Application Priority Data
Jun. 20, 2023 (CN) 202310733977.3

(51) Int. Cl. G16H 30/40; G06V 10/70; G06V 10/776; G06V 10/82; G06V 20/70

(52) U.S. Cl. CPC G16H 30/40; G06V 10/776; G06V 10/82; G06V 10/87; G06V 20/70

(57) ABSTRACT

The present disclosure provides a multi-modal multi-view classification method for echocardiograms based on a deep learning algorithm, including: collecting videos and images of multi-modal multi-view adult echocardiograms, and preprocessing the videos and images; annotating the preprocessed videos and images of adult echocardiograms to generate an adult echocardiogram dataset; dividing the adult echocardiogram dataset into a training set, a validation set, and a test set; constructing an adult echocardiogram view classification model based on a ResNet network, training the model using the training set, and selecting an optimal classification model using the validation set; evaluating performance of the optimal adult echocardiogram view classification model based on the test set; and inputting a to-be-tested image or video into the adult echocardiogram view classification model to obtain a classification result.

### CROSS REFERENCE TO RELATED APPLICATION

This patent application claims the benefit and priority of Chinese Patent Application No. 202310733977.3, filed with the China National Intellectual Property Administration on Jun. 20, 2023, the disclosure of which is incorporated by reference herein in its entirety as part of the present application.

### TECHNICAL FIELD

The present disclosure relates to the technical field of echocardiogram classification, and in particular, to a multi-modal multi-view classification method for echocardiograms based on a deep learning algorithm.

### BACKGROUND

Echocardiograms offer advantages such as non-invasiveness, being radiation-free, high temporal resolution, and bedside operability, playing a crucial clinical role in the diagnosis and treatment of cardiovascular diseases. Current echocardiograms include multiple modalities, such as M-mode, two-dimensional grayscale, color Doppler, and opacification. Multi-modal ultrasound images provide rich information about cardiac anatomy, such as the positions and sizes of the atriums and ventricles, as well as hemodynamic information. Accurate identification of standard views in echocardiograms is essential for quantification and post-processing analysis of cardiac functions. However, post-processing analysis software, such as QLab and EchoPAC, relies on manually selecting standard apical view images for secondary analysis of the cardiac structure and functions. For large sample data, pre-classification of views is time-consuming and labor-intensive. Therefore, the demand for rapid and accurate automatic identification of standard views in echocardiograms is gradually increasing.

In the prior art, identification of standard views in echocardiograms primarily focuses on 2D grayscale images, with limited research on 3D imaging and color Doppler ultrasound images. Therefore, it is highly necessary to design a multi-modal multi-view classification method for echocardiograms based on a deep learning algorithm.

### SUMMARY

An objective of the present disclosure is to provide a multi-modal multi-view classification method for echocardiograms based on a deep learning algorithm, to implement automatic multi-modal multi-view classification of echocardiograms with high accuracy, saving time and effort.

To achieve the above objective, the present disclosure provides the following technical solutions.

A multi-modal multi-view classification method for echocardiograms based on a deep learning algorithm is provided, including the following steps:
step 1: collecting videos and images of multi-modal multi-view adult echocardiograms;
step 2: preprocessing the collected videos and images of adult echocardiograms;
step 3: annotating the preprocessed videos and images of adult echocardiograms to generate an adult echocardiogram dataset;
step 4: dividing the adult echocardiogram dataset into a training set, a validation set, and a test set;
step 5: constructing an adult echocardiogram view classification model based on a ResNet network, training the model using the training set, and selecting an optimal classification model using the validation set;
step 6: evaluating the performance of the optimal adult echocardiogram view classification model based on the test set; and
step 7: inputting a to-be-tested image or video into the adult echocardiogram view classification model to obtain a classification result.

Optionally, in step 1, said collecting the videos and images of multi-modal multi-view adult echocardiograms specifically includes:
collecting the videos and images of multi-modal multi-view adult echocardiograms, including three-dimensional grayscale echocardiograms, color Doppler echocardiograms, two-dimensional grayscale parasternal left ventricular long-axis views, two-dimensional grayscale parasternal left ventricular short-axis views, two-dimensional grayscale parasternal aorta short-axis views, two-dimensional grayscale subxiphoid views, two-dimensional grayscale apical two-chamber views, two-dimensional grayscale apical three-chamber views, two-dimensional grayscale apical four-chamber views, two-dimensional apical two-chamber views based on left ventricular opacification, two-dimensional apical three-chamber views based on left ventricular opacification, two-dimensional apical four-chamber views based on left ventricular opacification, and two-dimensional parasternal left ventricular short-axis views based on left ventricular opacification.

Optionally, in step 2, said preprocessing the collected videos and images of adult echocardiograms specifically includes:
batch-processing the videos and images of adult echocardiograms using a Python third-party library OpenCV, extracting sector regions of interest based on pixel changes in consecutive frames by using image preprocessing operations, and saving the videos as frames in Portable Network Graphics (PNG) format.

Optionally, in step 4, said dividing the adult echocardiogram dataset into the training set, the validation set, and the test set specifically includes:
dividing the adult echocardiogram dataset into the training set, the validation set, and the test set in a ratio of 8:1:1. With view data having the smallest sample volume as a standard, datasets of other view categories are sampled at equal proportions to create a balanced dataset, ensuring balanced sample volumes for each view category. The training set is used to train the adult echocardiogram view classification model, the validation set is used to adjust model hyperparameters and select the optimal classification model, and the test set is used to evaluate the classification performance of the model.

Optionally: in step 5, said constructing the adult echocardiogram view classification model based on the ResNet network, training the model using the training set, and selecting the optimal classification model using the validation set specifically includes:
constructing the adult echocardiogram view classification model based on a 101-layer ResNet network: training the model using the training set, where a transfer learning strategy is employed during a training phase, training results on an ImageNet dataset are used as pre-trained weights, an output layer classifier uses a Softmax function, with 13 categories for classification, an Adam optimizer with an initial learning rate of 0.0001 is used, model fine-tuning is performed with a batch size of 128 for 100 iterations: after training, evaluating model performance on the validation set by minimizing a cross-entropy loss between real labels and predicted results; and selecting a model weight with highest classification accuracy as the optimal adult echocardiogram view classification model.

Optionally, in step 6, said evaluating the performance of the optimal adult echocardiogram view classification model based on the test set specifically includes:
evaluating the performance of the optimal adult echocardiogram view classification model on the validation set and the test set based on confusion matrix, accuracy, precision, recall, specificity, and F1 score.

Optionally, in step 7, said inputting the to-be-tested image into the adult echocardiogram view classification model to obtain the classification result specifically includes:
inputting the to-be-tested image into the adult echocardiogram view classification model, where for adult echocardiogram images, the adult echocardiogram view classification model predicts a classification result for each image, while for adult echocardiogram videos, the adult echocardiogram view classification model samples 10 frames from each video at regular intervals for prediction, takes an average value of predictions results, and uses a view category corresponding to a maximum prediction probability as a classification result for the video: based on a gradient-weighted class activation map visualization analysis method, generating a heatmap by using final-layer feature weights of the adult echocardiogram view classification model, to visualize focus areas of the view classification model, and performing an interpretable analysis on the classification result.

According to the embodiments of the present disclosure, the following technical effects are disclosed: The multi-modal multi-view classification method for echocardiograms based on a deep learning algorithm provided by the present disclosure includes the following steps: collecting videos and images of multi-modal multi-view adult echocardiograms: preprocessing the collected videos and images of adult echocardiograms: annotating the preprocessed videos and images of adult echocardiograms to generate an adult echocardiogram dataset: dividing the adult echocardiogram dataset into a training set, a validation set, and a test set: constructing an adult echocardiogram view classification model based on a ResNet network, training the model using the training set, and selecting an optimal classification model using the validation set: evaluating performance of the optimal adult echocardiogram view classification model based on the test set; and inputting a to-be-tested image or video into the adult echocardiogram view classification model to obtain a classification result. This method preprocesses videos and images of adult echocardiograms, extracts regions of interest, and obscures patient examination and personal information. The method can be applied to multi-modal (2D grayscale. Doppler. 3D. and left ventricular opacification) and multi-view (mainly for commonly used 2D grayscale clinical examination views) echocardiogram data. In the method, annotation is conducted by experienced ultrasound doctors, ensuring label accuracy. The method randomly divides the dataset in proportion, ensuring data diversity and sample balance. The method evaluates the prediction performance of the model on independent datasets through multiple classification metrics, ensuring the robustness of the prediction results of the model. The method uses a class activation heatmap to visualize focus anatomical areas of the model during the classification process, enhancing the interpretability of the model.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 is a schematic flowchart of a multi-modal multi-view classification method for echocardiograms based on a deep learning algorithm according to an embodiment of the present disclosure:

FIG. 2A is a diagram showing visual analysis results for view classes 2D_2C, 2D_3C, 2D_4C, and 2D_BX:

FIG. 2B is a diagram showing visual analysis results for view classes 2D_P, 2D_SAX, 2D_SAoA, 3D, C2D_2C, and C2D_3C; and

FIG. 2C is a diagram showing visual analysis results for view classes C2D_4C, C2D_SAX, and CDFI.

### DETAILED DESCRIPTION OF THE EMBODIMENTS

An objective of the present disclosure is to provide a multi-modal multi-view classification method for echocardiograms based on a deep learning algorithm, to implement automatic multi-modal multi-view classification of echocardiograms with high accuracy, saving time and effort.

In order to make the above objective, features, and advantages of the present disclosure clearer and more comprehensible, the present disclosure will be further described in detail below in combination with accompanying drawings and particular implementation modes.

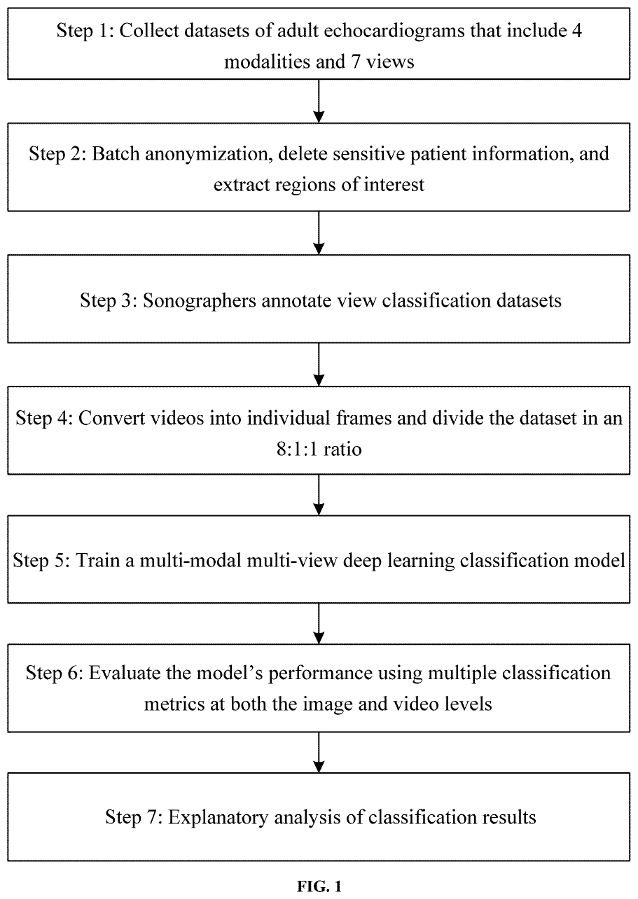

As shown in FIG. 1, the multi-modal multi-view classification method for echocardiograms based on a deep learning algorithm according to the embodiment of the present disclosure includes the following steps:

Step 1: Collect videos and images of multi-modal multi-view adult echocardiograms.

Step 2: Preprocess the collected videos and images of adult echocardiograms.

Step 3: Annotate the preprocessed videos and images of adult echocardiograms to generate an adult echocardiogram dataset.

Step 4: Divide the adult echocardiogram dataset into a training set, a validation set, and a test set.

Step 5: Construct an adult echocardiogram view classification model based on a ResNet network, train the model using the training set, and select an optimal classification model using the validation set.

Step 6: Evaluate the performance of the optimal adult echocardiogram view classification model based on the test set.

Step 7: Input a to-be-tested image or video into the adult echocardiogram view classification model to obtain a classification result.

In step 1, said collecting the videos and images of multi-modal multi-view adult echocardiograms is specifically as follows:

The videos and images of multi-modal multi-view adult echocardiograms are collected, including three-dimensional grayscale echocardiograms, color Doppler echocardiograms, two-dimensional grayscale parasternal left ventricular long-axis views, two-dimensional grayscale parasternal left ventricular short-axis views, two-dimensional grayscale parasternal aorta short-axis views, two-dimensional grayscale subxiphoid views, two-dimensional grayscale apical two-chamber views, two-dimensional grayscale apical three-chamber views, two-dimensional grayscale apical four-chamber views, two-dimensional apical two-chamber views based on left ventricular opacification, two-dimensional apical three-chamber views based on left ventricular opacification, two-dimensional apical four-chamber views based on left ventricular opacification, and two-dimensional parasternal left ventricular short-axis views based on left ventricular opacification.

In step 2, said preprocessing the collected videos and images of adult echocardiograms is specifically as follows:

Ultrasound video data is stored in Digital Imaging and Communications in Medicine (DICOM) format, usually containing redundant information such as examination information (examination date and time, electrocardiogram, heart rate, frame rate), and ultrasound equipment parameters. Such information may reduce the signal-to-noise ratio of the images and affect the prediction performance of AI models. The images and videos of adult echocardiograms are batch-processed using Python third-party libraries such as Pydicom and OpenCV. Based on pixel changes in consecutive frames, sector regions of interest are extracted with image preprocessing operations like dilation and erosion. The videos are saved as frames in PNG format for subsequent training of the view classification model. The original DICOM files are processed using the Pydicom library: while sensitive information such as patient names are removed. Regions of interest (sector regions) are extracted from the echocardiogram videos using the OpenCV library, while surrounding irrelevant information (such as patient age, name, and machine model) is eliminated.

In step 3, said annotating the preprocessed videos and images of adult echocardiograms to generate the adult echocardiogram dataset is specifically as follows:

Ultrasound doctors with rich clinical experience complete the annotation task for the view dataset, ensuring the authenticity and reliability of the data labels.

In step 4, said dividing the adult echocardiogram dataset into the training set, the validation set, and the test set is specifically as follows:

The adult echocardiogram dataset is divided into the training set, the validation set, and the test set in a ratio of 8:1:1. With view data having the smallest sample volume as a standard, datasets of other view categories are sampled at equal proportions to create a dataset with balanced sample volumes for each view category. The training set is used to train the adult echocardiogram view classification model, the validation set is used to adjust model hyperparameters and select the optimal classification model, and the test set is used to evaluate the classification performance of the model.

In step 5, said constructing the adult echocardiogram view classification model based on the ResNet network, training the model using the training set, and selecting the optimal classification model using the validation set is specifically as follows:

A convolutional neural network has powerful feature representation capabilities, typically composed of convolutional kernels, activation functions, pooling layers, and fully connected layers. A convolution operation can extract features from different levels of an input image. The activation function enhances feature recognition by introducing nonlinear relationships between network layers. The pooling layer reduces feature dimensions while retaining spatial and scale invariance of main image features. The fully connected layer maps the extracted features to a classification sample space. However, as the number of network layers increases, simply stacking convolutional layers may lead to the vanishing or exploding gradient problem, reducing model performance. The deep residual network ResNet is one of the most groundbreaking convolutional neural networks in the field of computer vision. It addresses the issue of degradation in deep network layers during the backward propagation process by reconstructing the model through residual learning and fitting residual mappings, significantly increasing network depth and effectively improving network performance.

The basic idea of ResNet is as follows: given an input x. H(x) represents learned features, that is, features extracted by nonlinear layers formed by several stacked convolutional layers. Another path is linear layers, representing identity mappings or projection, with learned residual features being F(x)=H(x)−x. Thus, the original mapping becomes F(x)+x, allowing the stacked convolutional layers to learn new features based on the input features, increasing the network depth and enhancing model performance.

In the present disclosure, the adult echocardiogram view classification model is constructed based on a 101-layer ResNet network. The model is trained using the training set. A transfer learning strategy is employed during a training phase. Training results on an ImageNet dataset are used as pre-trained weights. An output layer classifier uses a Softmax function, with 13 categories for classification. An Adam optimizer with an initial learning rate of 0.0001 is used, and model fine-tuning is performed with a batch size of 128 for 100 iterations. A convolutional neural network with residual structures is used to extract ultrasound image features. The specific process is as follows: an original-sized ultrasound image is inputted first, and is resized to 256*256*3 using the transforms. Resize ( ) method in PyTorch. In the training set, the transforms. RandomRotation ( ) method is applied to randomly rotate the image in a horizontal direction within a range of 0-15 degrees to enhance data diversity and robustness, thereby improving the generalization capability of the model. To enhance the convergence speed, stability, and generalization capability of the model, the transforms. Normalize ( ) method is applied to standardize images in the training set and validation set. Mean values for three image channels are set to 0.07, and standard deviations are 0.15, 0.15, and 0.14, respectively. The preprocessed image is inputted into a 7×7 kernel for convolution operations with a stride of 2, 64 output channels, and border padding of 3. Batch normalization and ReLU activation functions are then applied. Max-pooling with a 3×3 pooling kernel and a stride of 2 is performed to reduce the size of the feature map. Subsequently, a basic block group 1 is applied, consisting of three basic block structures. Each block includes two 1×1 convolutional kernels (with a stride of 1, and 64 and 256 output channels respectively) and one 3×3 convolutional kernel (with a stride of 1, 64 output channels, and border padding of 1). Batch normalization follows each convolutional kernel, and ReLU activation is applied after the second 1×1 convolutional kernel. Inputs of the three basic blocks bypass the convolutional layers via skip connections, and the inputs are directly added to the convolutional layer output, preventing the gradient vanishing problem. Three structures similar to the basic block group 1 are then applied, with output channels increasing from 256 to 512, 1024, and 2048, respectively. Global average pooling is applied to transform the feature map into a 2048*1*1 vector. The final output layer uses linear fully connected operations, and employs a Softmax function to map the feature vector to a probability distribution of 13 categories, for predicting a view category of the echocardiogram image.

After training, the performance of the model is evaluated on the validation set by minimizing a cross-entropy loss between real labels and predicted results. A model with a minimum loss value is selected as the optimal adult echocardiogram view classification model. Model development and validation are implemented using Python (version 3.7.10) and PyTorch (version 1.7.1) software on a server equipped with an NVIDIA Geforce RTX 3090 GPU (24 GB memory).

In step 6, said evaluating the performance of the optimal adult echocardiogram view classification model based on the test set is specifically as follows:

The performance of the optimal adult echocardiogram view classification model is evaluated on the validation set and the test set based on confusion matrix, accuracy: precision (also known as positive prediction value), recall (also known as sensitivity), specificity, and F1 score.

In step 7, said inputting the to-be-tested image or video into the adult echocardiogram view classification model to obtain the classification result is specifically as follows:

The to-be-tested image is inputted into the adult echocardiogram view classification model. For adult echocardiogram images, the adult echocardiogram view classification model predicts a classification result for each image. For adult echocardiogram videos, the adult echocardiogram view classification model samples 10 frames from each video at regular intervals for prediction, takes an average value of prediction results, and uses a view category corresponding to a maximum prediction probability as a classification result for the video. Based on a gradient-weighted class activation map visualization analysis method, a heatmap is generated by using final-layer feature weights of the adult echocardiogram view classification model, to visualize focus areas of the view classification model, and an interpretable analysis is performed on the classification result. This helps understand the deep logic of the view classification model and enhance the interpretability of the model.

An embodiment of the present disclosure involves training a multi-modal multi-view dataset with different classification models, evaluating the performance of the classification models in the image level of the validation set, and at the same time, evaluating the performance of the classification model at the image level and video level of the test set. The results are presented in Table 1.

TABLE 1
Prediction result of classification models in image level

| | Image level of validation set | | | Image level of test set | | |
|---|---|---|---|---|---|---|
| | top-1 | top-2 | F1 | top-1 | top-2 | F1 |
| Model | acc | acc | score | acc | acc | score |
| Densenet121 | 93.19 | 97.28 | 93.17 | 93.59 | 98.05 | 93.57 |
| Resnet18 | 93.00 | 97.65 | 92.96 | 96.27 | 99.39 | 95.98 |
| Resnet34 | 92.77 | 97.38 | 92.79 | 96.59 | 99.43 | 96.34 |
| Resnet50 | 92.84 | 97.18 | 92.79 | 96.43 | 99.20 | 96.15 |
| Resnet101 | 93.20 | 97.25 | 93.19 | 97.13 | 99.51 | 96.86 |
| Resnext50 | 92.99 | 97.56 | 92.99 | 96.83 | 99.58 | 96.53 |
| VGG-19 | 92.83 | 97.26 | 92.82 | 96.15 | 99.15 | 95.88 |
| ViT | 92.18 | 97.47 | 92.17 | 95.56 | 98.90 | 95.25 |
| Mobile_ViT | 92.50 | 97.59 | 92.51 | 92.69 | 97.94 | 92.09 |
| ConvNeXt | 92.94 | 97.46 | 92.84 | 96.47 | 99.29 | 96.13 |
| ShuffleNet | 92.93 | 97.84 | 92.95 | 96.21 | 99.23 | 95.93 |
| MobileNetV2 | 93.09 | 97.67 | 93.04 | 96.57 | 99.42 | 96.25 |

In the image level of the validation set, the performance of the model is evaluated using accuracy and F1 score. ResNet101 has the highest F1 score, indicating the optimal classification performance (see Table 1). Therefore, ResNet101 is selected as the final view classification model, and the classification performance of the model is evaluated in the image level and video level of the test set.

TABLE 2
Prediction results of ResNet101-based multi-modal multi-
view classification model in image level of test set

| View category | Precision | Recall | F1 score | Specificity |
|---|---|---|---|---|
| 2D_2C | 97.73 | 99.89 | 98.80 | 99.80 |
| 2D_3C | 99.89 | 97.46 | 98.66 | 100.00 |
| 2D_4C | 99.42 | 99.89 | 99.66 | 99.90 |
| 2D_BX | 99.95 | 99.16 | 99.55 | 100.00 |
| 2D_P | 99.02 | 99.62 | 99.32 | 99.90 |
| 2D_SAX | 98.70 | 99.37 | 99.03 | 99.90 |
| 2D_SAoA | 99.95 | 98.16 | 99.05 | 100.00 |
| 3D | 100.00 | 100.00 | 100.00 | 100.00 |
| C2D_2C | 88.29 | 84.35 | 86.28 | 99.20 |
| C2D_3C | 90.36 | 91.89 | 91.12 | 99.30 |
| C2D_4C | 91.69 | 93.36 | 92.52 | 99.30 |
| C2D_SAX | 94.47 | 95.94 | 95.20 | 99.60 |
| CDFI | 100.00 | 99.89 | 99.95 | 100.00 |
| Average value | 96.88 | 96.85 | 96.86 | 99.76 |

Note: 2D_2C represents a two-dimensional grayscale apical two-chamber view; 2D_3C represents a two-dimensional grayscale apical three-chamber view; 2D_4C represents a two-dimensional grayscale apical four-chamber view; 2D_BX represents a two-dimensional grayscale subxiphoid view; 2D_P represents a two-dimensional grayscale parasternal left ventricular long-axis view; 2D_SAX represents a two-dimensional grayscale parasternal left ventricular short-axis view; 2D_SAoA represents a two-dimensional grayscale parasternal aorta short-axis view; 3D represents a three-dimensional grayscale echocardiogram; C2D_2C represents a two-dimensional apical two-chamber view based on left ventricular opacification; C2D_3C represents a two-dimensional apical three-chamber view based on left ventricular opacification; C2D_4C represents a two-dimensional apical four-chamber view based on left ventricular opacification: C2D_SAX represents a two-dimensional parasternal left ventricular short-axis view based on left ventricular opacification: CDFI represents a color Doppler echocardiogram.

The results indicate that, in the image level. ResNet101 achieves an average accuracy of 97.13% and an average F1 score of 96.86 for the classification of 13 view categories (see Table 2). In the video level. ResNet101 attains an average F1 score of 97.07 for the classification of 13 view categories.

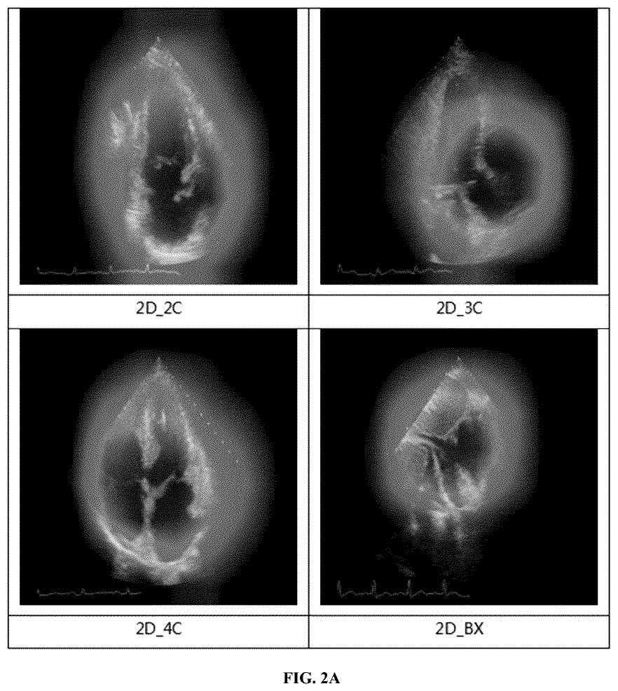

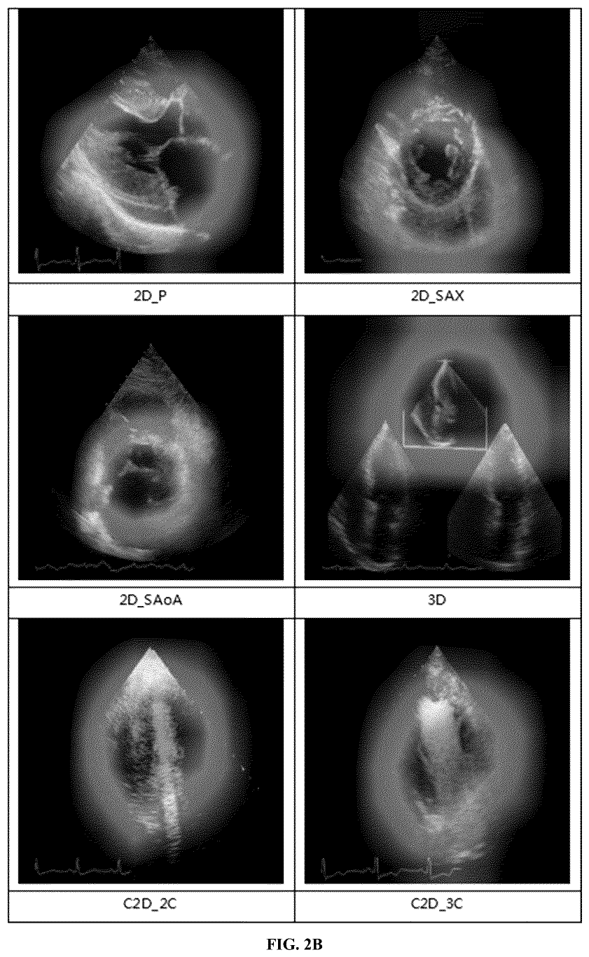

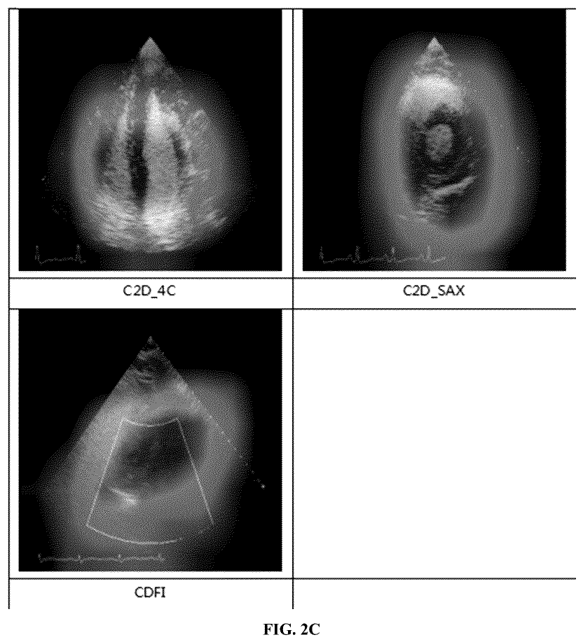

The interpretability analysis conducted on the test set using the Grad-CAM visualization method indicates that the focus areas used by the view classification model to determine image categories are consistent with those of ultrasound doctors. For example, as illustrated in FIGS. 2A-2C, for the two-dimensional grayscale apical four-chamber view: both the model and the doctor pay attention to the cross area of the atrium and ventricle.

The multi-modal multi-view classification method for echocardiograms based on a deep learning algorithm provided by the present disclosure includes the following steps: collecting videos and images of multi-modal multi-view adult echocardiograms: preprocessing the collected videos and images of adult echocardiograms: annotating the preprocessed videos and images of adult echocardiograms to generate an adult echocardiogram dataset: dividing the adult echocardiogram dataset into a training set, a validation set, and a test set: constructing an adult echocardiogram view classification model based on a ResNet network: training the model using the training set, and selecting an optimal classification model using the validation set: evaluating performance of the optimal adult echocardiogram view classification model based on the test set; and inputting a to-be-tested image or video into the adult echocardiogram view classification model to obtain a classification result. This method preprocesses videos and images of adult echocardiograms, extracts regions of interest, and obscures patient examination and personal information. The method can be applied to multi-modal (2D grayscale. Doppler. 3D. and opacification) and multi-view (mainly for commonly used 2D grayscale clinical examination views) echocardiogram data. In the method, annotation is conducted by experienced ultrasound doctors, ensuring label accuracy. The method randomly divides the dataset in proportion, ensuring data diversity and sample balance. The method evaluates the prediction performance of the model on independent datasets through multiple classification metrics, ensuring the robustness of the prediction results of the model. The method uses a class activation heatmap to visualize focus anatomical areas of the model during the classification process, enhancing the interpretability of the model.

Particular examples are used herein for illustration of principles and implementation modes of the present disclosure. The descriptions of the above embodiments are merely used for assisting in understanding the method of the present disclosure and its core ideas. In addition, those of ordinary skill in the art can make various modifications in terms of particular implementation modes and the scope of application in accordance with the ideas of the present disclosure. In conclusion, the content of the description shall not be construed as limitations to the present disclosure.

1. A multi-modal multi-view classification method for echocardiograms based on a deep learning algorithm, comprising the following steps:
step 1: collecting videos and images of multi-modal multi-view adult echocardiograms;
step 2: preprocessing the collected videos and images of adult echocardiograms;
step 3: annotating the preprocessed videos and images of adult echocardiograms to generate an adult echocardiogram dataset;
step 4: dividing the adult echocardiogram dataset into a training set, a validation set, and a test set;
step 5: constructing an adult echocardiogram view classification model based on a ResNet network, training the model using the training set, and selecting an optimal classification model using the validation set;
step 6: evaluating performance of the optimal adult echocardiogram view classification model based on the test set; and
step 7: inputting a to-be-tested image or video into the adult echocardiogram view classification model to obtain a classification result.

2. The multi-modal multi-view classification method for echocardiograms based on a deep learning algorithm according to claim 1, wherein in step 1, said collecting the videos and images of multi-modal multi-view adult echocardiograms specifically comprises:
collecting the videos and images of multi-modal multi-view adult echocardiograms, comprising three-dimensional grayscale echocardiograms, color Doppler echocardiograms, two-dimensional grayscale parasternal left ventricular long-axis views, two-dimensional grayscale parasternal left ventricular short-axis views, two-dimensional grayscale parasternal aorta short-axis views, two-dimensional grayscale subxiphoid views, two-dimensional grayscale apical two-chamber views, two-dimensional grayscale apical three-chamber views, two-dimensional grayscale apical four-chamber views, two-dimensional apical two-chamber views based on left ventricular opacification, two-dimensional apical three-chamber views based on left ventricular opacification, two-dimensional apical four-chamber views based on left ventricular opacification, and two-dimensional parasternal left ventricular short-axis views based on left ventricular opacification.

3. The multi-modal multi-view classification method for echocardiograms based on a deep learning algorithm according to claim 2, wherein in step 2, said preprocessing the collected videos and images of adult echocardiograms specifically comprises:
batch-processing the videos and images of adult echocardiograms using a Python third-party library OpenCV, extracting sector regions of interest based on pixel changes in consecutive frames by using image preprocessing operations, and saving the videos as frames in Portable Network Graphics (PNG) format.

4. The multi-modal multi-view classification method for echocardiograms based on a deep learning algorithm according to claim 3, wherein in step 4, said dividing the adult echocardiogram dataset into a training set, a validation set, and a test set specifically comprises:
dividing the adult echocardiogram dataset into the training set, the validation set, and the test set in a ratio of 8:1:1, wherein with view data having a smallest sample volume as a standard, datasets of other view categories are sampled at equal proportions to create a dataset with balanced sample volumes for each view category; the training set is used to train the adult echocardiogram view classification model, the validation set is used to adjust model hyperparameters and select the optimal classification model, and the test set is used to evaluate classification performance of the model.

5. The multi-modal multi-view classification method for echocardiograms based on a deep learning algorithm according to claim 4, wherein in step 5, said constructing the adult echocardiogram view classification model based on the ResNet network, training the model using the training set, and selecting the optimal classification model using the validation set specifically comprises:
constructing the adult echocardiogram view classification model based on a 101-layer ResNet network; training the model using the training set, wherein a transfer learning strategy is employed during a training phase, training results on an ImageNet dataset are used as pre-trained weights, an output layer classifier uses a Softmax function, with 13 categories for classification, an Adam optimizer with an initial learning rate of 0.0001 is used, model fine-tuning is performed with a batch size of 128 for 100 iterations, and a convolutional neural network with residual structures is used to extract ultrasound image features; after training, evaluating model performance on the validation set by minimizing a cross-entropy loss between real labels and predicted results; and selecting a model weight with highest classification accuracy as the optimal adult echocardiogram view classification model.

6. The multi-modal multi-view classification method for echocardiograms based on a deep learning algorithm according to claim 5, wherein in step 6, said evaluating the performance of the optimal adult echocardiogram view classification model based on the test set specifically comprises:
evaluating the performance of the optimal adult echocardiogram view classification model on the validation set and the test set based on confusion matrix, accuracy, precision, recall, specificity, and F1 score.

7. The multi-modal multi-view classification method for echocardiograms based on a deep learning algorithm according to claim 6, wherein in step 7, said inputting the to-be-tested image or video into the adult echocardiogram view classification model to obtain the classification result specifically comprises:
inputting the to-be-tested image into the adult echocardiogram view classification model, wherein for adult echocardiogram images, the adult echocardiogram view classification model predicts a classification result for each image, while for adult echocardiogram videos, the adult echocardiogram view classification model samples 10 frames from each video at regular intervals for prediction, takes an average value of predictions results, and uses a view class corresponding to a maximum prediction probability as a classification result for the video; based on a gradient-weighted class activation map visualization analysis method, generating a heatmap by using final-layer feature weights of the adult echocardiogram view classification model, to visualize focus areas of the view classification model, and performing an interpretable analysis on the classification result.
